(19) United States
(12) Patent Application Publication
(10) Pub. No.: US 2013/0130299 A1
(43) Pub. Date: May 23, 2013

(54) ELECTROCHEMICAL FLOW CYTOMETRY

(71) Applicant: University of Utah Research Foundation, Salt Lake City, UT (US)

(72) Inventors: Gregory Dittami, Salt Lake City, UT (US); Richard Rabbitt, Salt Lake City, UT (US)

(73) Assignee: University of Utah Research Foundation, Salt Lake City, UT (US)

(21) Appl. No.: 13/739,800

(22) Filed: Jan. 11, 2013

Related U.S. Application Data
(62) Division of application No. 12/558,281, filed on Sep. 11, 2009.

(51) Int. Cl. C12Q 1/02

(52) U.S. Cl. CPC C12Q 1/02
USPC 435/29

(57) ABSTRACT

A method for triggering cellular exocytosis events and measuring quantal release of electroactive chemicals is disclosed. A method can include flowing a cell through a single cell channel having a pair of electrodes oriented to direct current through the cell at a stimulation location along the single cell channel. The pair of electrodes include a working electrode, a counter electrode and an optional reference electrode. The single cell channel has dimensions which provide direct contact of the cell with walls of the single cell channel sufficient to substantially reduce or eliminate shunt paths around the cell at the stimulation location. The cell can be exposed to an electric field at the stimulation location using the pair of electrodes sufficient to trigger exocytosis. Changes in current due to exocytosis processes appear as spikes that can be correlated with a quanta of the electroactive chemicals which are released during exocytosis.

### RELATED APPLICATION(S)

This application is a divisional application of U.S. patent application Ser. No. 12/558,281 filed Sep. 11, 2009, which is incorporated herein by reference.

### GOVERNMENT INTEREST

This invention was made with government support under Grant #DC004928 awarded by the National Institutes of Health and Grant #DGE9987616 awarded by the National Science Foundation. The Government has certain rights to this invention

### BACKGROUND

Controlled exocytosis of biochemical transmitters is the principal means of communication utilized by neuronal cells and secretory endocrine cells. Certain congenital disorders, toxins/drugs, and diseases can alter exocytotic events and thereby lead to serious physiological problems. Consequently, studies of the underlying mechanisms of exocytosis are important to our basic understanding of how this mechanism is affected as well as to the development of potential therapeutic interventions. One approach to studying exocytosis is to place a carbon fiber electrode adjacent to the cell membrane of a stationary cell in close vicinity to the release site and to electrochemically detect single vesicle, or quantal release. The power of this technique lays in the high temporal resolution and sensitivity of detection with the ability to resolve microsecond scale events and zeptomole quantities of released neurotransmitter, respectively. Since its inception, this method has been extensively applied to studies of cellular exocytosis.

Conventional electrochemical detection of transmitter release immobilizes the cell and uses a micromanipulator to position a carbon fiber electrode (CFE) in direct contact with the plasma membrane. Release is evoked by application of chemical stimuli or by depolarization of the cell using whole-cell voltage/current clamp. This approach requires individualized clamping of each cell for detection and operation.

Recently, a number of groups have advanced standard electrochemistry technology by leveraging micro-electromechanical systems (MEMS) fabrication techniques. With MEMS, it is possible to achieve microfluidic and electrode-array architectures for facilitated selection and precise positioning of cells relative to the electrodes of interest. Currently, the majority of MEMS-implemented detection schemes involve the culture or seeding of cells on top of solitary electrodes or electrode arrays. This approach has the advantage of ensuring direct contact of cells with the electrode(s) of interest. Furthermore, as the cells are immobilized, often through adhesion-promoters such as poly-lysine, it is straightforward to deliver solutions to the cells for depolarization, differentiation and/or pharmacology, without displacing the cells. An alternative approach is to use microfluidic-based MEMS to position cells. With both designs, cells were customarily stimulated using depolarizing solutions of elevated potassium, nicotine, or barium chloride. This requires a separate fluidic infrastructure to consistently and rapidly deliver and exchange solutions to the cell and methods to account for the time delay between solution delivery and depolarization. Photo-released caged Ca²⁻ has also been used to stimulate chromaffin cells and avoid some of the challenges associated with chemical delivery systems. However, one drawback of this approach is that it required a permeabilizing and pre-loading of the cells with the caged Ca²⁺. Therefore, although somewhat effective, current approaches have drawbacks which limit their widespread use and effectiveness, particularly with respect to higher-throughput studies of exocytosis.

### SUMMARY

The present disclosure provides a method for triggering cellular exocytosis events and measuring quantal release of electroactive chemicals. A method can include flowing a cell or group of cells through a single cell microchannel having a pair of electrodes connected to a potentiostat circuit and oriented to direct current through the cell at a stimulation location along the single cell channel. The pair of electrodes includes a working electrode and a counter electrode in which the counter electrode is used to inject current to maintain the working electrode at a sufficient potential to oxidize or reduce the electroactive chemicals of interest. An optional third, reference electrode, can be used to measure the precise potential of the cell solution against which the working electrode voltage is controlled. The single cell channel has dimensions which provide direct contact of the cell with walls of the single cell channel sufficient to substantially reduce or eliminate shunt paths around the cell at the stimulation location. The cell can be exposed to an electric field generated at the stimulation location for detection of released electrochemicals. Simultaneously, current is measured at the working electrode. The total measured current is comprised of the current necessary to generate the specified working electrode potential and additional current generated as electroactive chemicals are oxidized or reduced at the electrode resulting in an electron transfer reaction with the electrode. The changes in current due to the latter processes appear as spikes that can be correlated with a quanta of the electroactive chemicals which are released during exocytosis. Flow can be continuous or discontinuous depending on the cell and desired conditions.

In practicing the above method, a unique electrochemical flow cytometer can be used. The electrochemical flow cytometer can include a single cell channel having an inlet and an outlet. The channel has a diameter which is equal to or less than a free diameter of the cell. A pair of electrodes is oriented along the single cell channel to force a current through the cell at a stimulation location along the single cell channel. The pair of electrodes are configured for both electrochemical stimulation and measurement of exocytosis events.

There has thus been outlined, rather broadly, certain features of the invention so that the detailed description thereof that follows may be better understood, and so that the present contribution to the art may be better appreciated. Other features of the present disclosure will become clearer from the following detailed description, taken with the accompanying drawings and claims, or may be learned by the practice of the invention.

### DESCRIPTION OF THE DRAWINGS

FIG. 1 is a top view of an electrochemical flow cytometer in accordance with one embodiment.

FIG. 2 is a side view of an electrochemical flow cytometer in accordance with one embodiment.

FIG. 3A is an optical micrograph of the MEMS microchip recording chamber in accordance with another embodiment, which is labeled: (i) fluidic channel (10 μm deep), (ii) two of the electrodes in the interdigitated array and (iii) a PC12 cell being pulled into the chamber. Also labeled are the working electrode (5 μm×10 μm, WE) and counter electrode (CE) used in the potentiostat circuit. A separate Ag/AgCl reference electrode (not shown) is located further downstream.

FIG. 3B is a magnified view of FIG. 3A where the cell is isolated over the WE via a 5 μm constriction in the channel ensuring direct contact of the cell with the WE. Electrodes on either side of the cell can, optionally, be connected to high-impedance amplifiers for passive measurement of voltage across the cell (as shown).

FIG. 4 is a graph of current versus time showing quantal events in accordance with an embodiment of the present disclosure. It is one example current trace recorded during amperometric (+700 mV vs Ag/AgCl) measurement of release from a cluster of six cells in the recording chamber. (INSET) (i) A typical recorded current spike corresponding to a quantal release event. (ii) In a subset of spikes, “foot” events (indicated by arrow) were detected consistent with the fusion pore formation hypothesis. (iii) Example of a “flicker” event which has been interpreted as resulting from closure of the fusion pore prior to full fusion of the synaptic vesicle with the plasma membrane.

FIG. 5A is a bin histogram for a population of dexamethasone (5 μM)-treated PC12 cells (n=209 cells, k=25,463 spikes) showing peak height in accordance with an embodiment of the present disclosure.

FIG. 5B is a bin histogram using the data of FIG. 5A to show t_{1/2}.

FIG. 5C is a bin histogram using the data of FIG. 5A showing the cube root of quantal molar size. Fitting of the curves with double Gaussian functions suggested the detection of spikes from multiple release sites of the cells. Means (μ) and standard deviations (σ) for the Gaussian fits are also shown in each of FIG. 5A through 5C.

FIG. 6A through 6D correlates several properties with cell size in accordance with an embodiment of the present disclosure. Bars show the mean (±1 standard error) for various spike parameters as a function of cell size. Note: “# of spikes” (a) represents the number of quantal release events in the first 60 seconds of release. The four cell sizes (n=sample size) categories were: C=Clusters (two or more cells, n=61), L=Large (n=76), M=Medium (n=34), S=Small (n=10).

FIGS. 7A and 7B are FEM simulations to study effect of cell size in accordance with an embodiment of the present disclosure. These images show the distributions of the voltages in the channel for a small cell (FIG. 7A) and large cell (FIG. 7B) for an applied WE voltage (DC, “δv” denoted voltage level in the channel). The scale, indicating the fractional voltage distribution, highlights the electric field patterning technique (μ-domain voltage clamp). Relative to the extracellular fluid, the membrane impedance is very high resulting in the majority of the applied voltage dropping across the cell. The tighter fit of the larger cell creates more depolarization simply due to the restriction of the extracellular shunt path.

FIG. 8 is a graph showing pulse evoked release in accordance with an embodiment of the present disclosure. For small cells that were not sufficiently depolarized by potentiostat electric field, release could occasionally be triggered through the application of an extracellular voltage pulse. The graph shows the working electrode current trace that shows onset of release following application of a voltage pulse (i).

FIG. 9 is a graph showing exocytosis flow cytometry in accordance with an embodiment of the present disclosure. Results for three separate cells flowing along the channel and crossing the WE in sequence are indicated at points marked i, ii, and iii respectively. Lower trace shows the working electrode current and the upper trace shows the voltage drop recorded across the cell. Current spikes, corresponding to quantal release events, are detectable for all three cells, with a visible correlation between the spike intensity and the magnitude of (negative) change in ΔV across the cell.

It will be understood that the drawings are provided merely for convenience in understanding the following detailed description. Deviation in proportions, dimensions, and other illustrated features can be had without departing from the scope of the disclosure.

### DETAILED DESCRIPTION OF EXEMPLARY EMBODIMENTS

While these exemplary embodiments are described in sufficient detail to enable those skilled in the art to practice the invention, it should be understood that other embodiments may be realized and that various changes to the invention may be made without departing from the spirit and scope of the present disclosure. Thus, the following more detailed description of the embodiments of the present disclosure is not intended to limit the scope of the invention, as claimed, but is presented for purposes of illustration only and not limitation to describe the features and characteristics of the present invention, to set forth the best mode of operation of the invention, and to sufficiently enable one skilled in the art to practice the invention. Accordingly, the scope of the present invention is to be defined solely by the appended claims.

Definitions

In describing and claiming the present invention, the following terminology will be used.

The singular forms “a,” “an,” and “the” include plural referents unless the context clearly dictates otherwise. Thus, for example, reference to “a cell” includes reference to one or more of such materials and reference to “subjecting” refers to one or more such steps.

As used herein with respect to an identified property or circumstance, “substantially” refers to a degree of deviation that is sufficiently small so as to not measurably detract from the identified property or circumstance. The exact degree of deviation allowable may in some cases depend on the specific context.

As used herein, “adjacent” refers to the proximity of two structures or elements. Particularly, elements that are identified as being “adjacent” may be either abutting or connected. Such elements may also be near or close to each other without necessarily contacting each other. The exact degree of proximity may in some cases depend on the specific context.

As used herein, “free diameter” refers to the average diameter of a cell in an undisturbed and non-deformed state.

As used herein, “single cell channel” refers to a channel through which a cell (or cells) passes through single-file (i.e. a single cell across). In some cases, individual cells will pass through the channel and/or constriction one at a time. In other cases, a cluster of cells can be deformed into a single-file train of cells as it passes through the channel and/or constriction. Such clusters may or may not return to their original shape depending on the interfacial strength between cells.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a numerical range of about 1 to about 4.5 should be interpreted to include not only the explicitly recited limits of 1 to about 4.5, but also to include individual numerals such as 2, 3, 4, and sub-ranges such as 1 to 3, 2 to 4, etc. The same principle applies to ranges reciting only one numerical value, such as “less than about 4.5,” which should be interpreted to include all of the above-recited values and ranges. Further, such an interpretation should apply regardless of the breadth of the range or the characteristic being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

### Embodiments

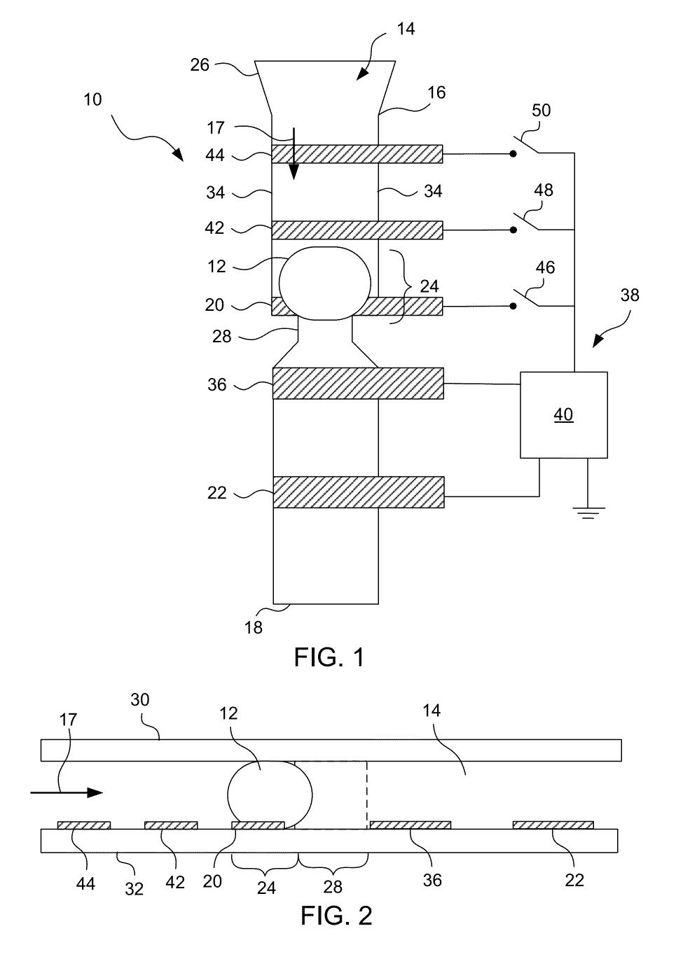

FIG. 1 shows an electrochemical flow cytometer 10 for measuring exocytosis events from a cell 12. The cytometer has a single cell channel 14 with an inlet 16 and an outlet 18. The channel has a diameter which is equal to or less than a free diameter of the cell. A pair of electrodes can include a working electrode 20 and a counter electrode 22. These electrodes are oriented along the single cell channel to force a current through the cell at a stimulation location 24 (typically over the working electrode) along the single cell channel. The pair of electrodes is configured for both electrochemical stimulation and measurement of exocytosis events as described in more detail below. An additional reference electrode 36 can be used to precisely measure the solution potential against which the working electrode potential is maintained.

The channel 14 has dimensions which admit only a single cell or a single-file cluster of cells at the inlet 16 along flow direction 17. Although not required, the inlet can be preceded by a tapering inlet section 26 which allows gradual reduction in cross-section to the desired single-cell path. In FIG. 1, a constriction 28 is illustrated immediately adjacent the stimulation location. In practice, the constriction acts to slow the cell movement, as well as to further engage the cell outer walls against the periphery walls of the channel to block shunt paths between the counter electrode 22 and the working electrode 20. Alternatively, or in addition, the top wall 30 and bottom wall 32 (shown in FIG. 2), as well as side walls 34 can be spaced so as to compress the cell and substantially reduce or eliminate fluid space between the walls and the cell. In this manner, applied current across the pair of electrodes will be forced to pass through the cell rather than to shunt around the cell or along its surface while the cell is at the stimulation location.

The side walls and channel can be formed of any suitable material. Typically, the material can be non-conductive, and non-limiting examples of such material can include silicon, polydimethylsiloxane, polyimide, polysilicon, glass (silica), and commercially available photopolymers such as the SU-8™ series photoresists (Microchem Corp, Newton, Mass.) or AZ P4000™ series photoresists (AZ Electronics Materials Corp, Charlotte, N.C.). Generally, biologically compatible materials are particularly suitable so as to not adversely affect the cells. Further, optional coatings can be used along internal surfaces of the channel in order to reduce adherence of cells or other debris. Non-limiting examples of suitable coatings can include fluoro-based coatings such as Pluronic F-108 and protein coatings such as bovine serum albumin. An optional filter can also be placed at the inlet to the channel to prevent materials larger than the cells from entering the channel/constriction and potentially clogging the channel.

Although the dimensions of the channel can vary, the channel can have a cross-sectional diameter equal to or less than a free diameter of the cell (i.e. the cell is in a compressed state). The dimensions of the channel can be sufficiently constrained to allow the cell to completely block the channel at the stimulation location. Thus, the channel can be gradually tapered, step-wise reduction, or have a constant cross-section along the path. Regardless of the specific shape, the cross-sectional diameter is sufficiently small to prevent an applied current from bypassing or shunting past the cell rather than being forced through the cell. This can generally be accomplished by sizing the channel to provide substantially complete fluidic isolation of the working electrode from the counter electrode when the cell is at the stimulation location. Any dimension equal to or smaller than the free diameter can be suitable, although a cross-sectional diameter from about 60% to about 90% of the free diameter can be particularly suitable. The actual dimension can depend on the cell size. Broadly, the free diameter of most cells is from about 5 μm to about 30 μm for Eukaryotic cells, and most often from about 10 μm to about 20 μm. In several specific examples, the free diameter of rat pheochromocytoma (PC12) cells and bovine chromaffin cells, can be 9-12 μm and 15-20 μm, respectively.

Because the pair of electrodes provides both stimulation and measurement functionality there is no need for other electrodes on a separate circuit. Thus, in one embodiment, the cytometer has only a single set of electrodes connected in a common circuit and configured to apply current across the channel. A variety of electrode arrangements can be had which include at least the working electrode and the counter electrode. Typically, the counter electrode 22 is oriented downstream from the stimulation location 24, and optionally also downstream of the working electrode 20. The counter electrode can be designed to be larger than the working electrode to ensure that reactions at the surface of the working electrode are not current-limited due to the surface area, and correspondingly the current injecting capabilities, of the counter electrode. When used, the reference electrode is typically positioned between the working and counter electrodes but can be located peripherally in contact with the solution media as well. The counter and working electrodes can be formed of any chemically-inert, highly polarizable electrically conductive material such as, but not limited to, platinum, gold, indium tin oxide and carbon. The reference electrode is preferably formed from a non-polarizable material such as silver/silver chloride or saturated calomel. Surface treatment of the working electrode in particular can be used to prevent non-specific chemical detection such as with Nafion coatings used for dopamine detection. Surface treatments can additionally be applied to preempt or minimize working electrode fouling such as through the use of mercaptopropionic acid in the detection of dopamine. The electrodes can generally span the channel width, although other shapes can be suitable. In one specific embodiment, a reference electrode 36 is oriented between the counter electrode and the working electrode to form a potentiostat circuit 38 including a potentiostat controller 40. The specific orientation of each electrode can be varied. In one aspect, the counter electrode and the working electrode can be oriented on a common side of the channel as illustrated in FIG. 1, and most clearly in FIG. 2. Optionally, the electrodes can be oriented on opposing sides of the channel as long as the current is directed through the cell at the stimulation location.

One or more supplementary working electrodes 42 and 44 can be oriented upstream of the working electrode 20. These supplementary electrodes can be used as alternatives to the primary working electrode 24. In some cases it can be desirable to use these electrodes as back-up electrodes such as when the primary electrode becomes damaged or fouled sufficient to prevent accurate readings or clear stimulation. In such cases, switches 46, 48 and 50 can be switched ‘on’ when the corresponding electrode is desired, while the other switches are left in the ‘off position (as illustrated).

The flow cytometer can be integrated into a suitable form factor such as, but not limited to, a stand-alone, handheld or benchtop unit. In one alternative, the system described above would serve as a disposable core of the unit. In this regard, a cartridge architecture can be employed where chips are rapidly exchanged as electrodes wear and foul over the course of use. The unit itself can be similar to those described as the “interface” in Dittami et al., Journal of Microelectromechanical Systems, 2008, 17, pp. 850-862 , which is incorporated herein by reference. For standard operation, the user loads a new chip into the unit, dispense a volume of cells into an entry well/reservoir or access point, and then activate a pump to pull the cells through the channel. The system would then perform the described stimulation and recording of exocytosis from the cells and provide a display (i.e. graphs and charts) summarizing the values and statistics for relevant exocytosis parameters including percentage of cells firing, quantal size, half-widths, peak heights, spike frequency and number of events per cell.

A method of triggering cellular exocytosis events and measuring quantal release of electroactive chemicals based on the above concepts can include flowing a cell or cluster of cells through a single cell channel. The single cell channel includes a pair of electrodes oriented to direct current through the cell at a stimulation location along the single cell channel. The pair of electrodes includes a working electrode and a counter electrode as previously described. The single cell channel has dimensions which provide direct contact of the cell with walls of the single cell channel sufficient to substantially reduce or eliminate shunt paths around the cell at the stimulation location.

The channel is a flow-through channel which allows continuous flow past the stimulation location. A suitable micropump can be oriented upstream or downstream of the channel in order to force fluid, including at least one cell, through the channel. Typically, a population of cells is prepared so as to provide a known population. The population is delivered to the channel such that individual cells are directed one at a time past the stimulation location. The geometry and/or flow can be adjusted to allow for a temporary pause of the cell at the recording location during the stimulation and detection. For example, the flow can be pulsed sufficient to temporarily allow the cell to stop at the stimulation location. Alternatively, a physical barrier can be incorporated into the channel wall such as a constriction (as illustrated in FIG. 1, feature 28). In this case, the cell is highly deformable and can squeeze through the constriction, but only after a brief pause during which pressure builds up around the cell sufficient to force the cell through the constriction. The pressure can be positive or negative pressure, depending on where the pump is located. In another alternative, the flow of the cell or cells is substantially continuous. In the latter case, the exposure time of the cell to a stimulating electric field is generally very small (e.g. from about 100 msec to about 1 sec).

More particularly, the cell can be exposed to an electric field at the stimulation location using the pair of electrodes sufficient to trigger exocytosis. Advantageously, the pair of electrodes can operate with a substantially continuous electric field. Thus, as the cell moves over the stimulation location, the electric field substantially ceases to go through the open channel and is forced through the cell. In one aspect, the working electrode is substantially completely fluidically-isolated from the counter electrode when the cell as located at the stimulation location in order to direct current through the cell rather than shunting around the cell. A majority of extracellular voltage drops across the cell, which depolarizes the cell and initiates exocytosis.

In one aspect, the electric field is a pulsed electric field. The frequency and magnitude of a pulsed electric field can be sufficient to augment the existing electric field to depolarize the cell while not exceeding values that would result in electroporation of the cell and cell death. The applied pulse can be administered from either a voltage source or a current source. The current source can minimize the channel voltage fluctuations due to the electrode double-layers ensuring a more consistent depolarizing voltage across the cell. Although the strength of the electric field can vary, typically the electric field can have an applied potential above an exocytosis threshold and below the water window. These limits can vary somewhat depending on the channel configuration, electrode composition and size, cell type, and surrounding cell environment. However, as a general guideline, an applied voltage from about −900 mV to about 1200 mV can be suitable. Under the conditions described herein it is possible to trigger exocytosis in a large percentage of the cells the pass through the channel. Generally, greater than about 50% and in some cases greater than 80% of the cells can be triggered.

Electrical current passing into the working electrode from the counter electrode and oxidation/reduction reactions can be measured and is typically integrated as part of the potentiostat controller. Transient changes in the measured current due to the oxidation/reduction reactions can then be correlated with a quanta of the electroactive chemicals. As can be appreciated from the example below, exocytosis events are typically associated with a spike in current as the electroactive compounds are released. Initially, the electro active compounds have an electric charge and are then quickly reduced or oxidized. As such, the total current measured at the working electrode will exhibit spikes, each of which can be associated with an exocytosis event. The magnitude of these spikes can be correlated with a known amount of the electroactive compound based on known baselines. Although the exact amounts of released chemicals can vary, the resolution of the approach described herein is very high and can readily measure quantities in the zeptomole range. Accurate readings have been achieved from about 100 zmole to about 1 amole. Thus, the spike can be associated with a single vesicle quantal release event and the shape of the current spike correlated to the release event can be used to characterize vesicle fusion kinetics. The resulting data can also be used to collect population statistics for identifying the role of drug interactions and/or knockout studies of proteins which involve exocytotic processes. For example, amperometric analysis of quantal release events can be used to determine drug efficacy.

The electroactive chemicals can be any materials which are released from a cell via electrically induced exocytosis. Non-limiting examples of such electroactive chemicals include neurotransmitters (e.g. dopamine, serotonin, epinephrine, norepinephrine, histamine, DOPAC, HVA, Nitric Oxide, ascorbic acid and 5-HIAA), and the like.

Over time, the working electrode can become contaminated with deposits of proteins or other residue. These deposits can be cleaned from the working electrode by cycling its potential over a period of time. For example voltage can be cycled across the working electrode from −300 mV to +1500 mV vs. Ag/AgCl in a 50-100 mM sulfuric acid solution or from −200 mV to −1200 mV vs Ag/AgCl in a 50 mM potassium hydroxide (KOH) solution. Although time can vary, typically at least 10 cycles at a rate of 50 mV/sec to 1 V/sec can be sufficient depending on the degree of fouling. Alternatively, electrodes can be cleaned by immersion in solutions of organic cleansing solutions such as mild piranha etch (50 mM H₂SO₄, 25% H₂O₂) for 5-10 minutes.

The flow cytometers can be formed using any suitable approach. Due to the small sizes, semiconductor fabrication processes and surface micromachining can be useful. Alternatively, the cytometers can be fabricated using thick-film processing techniques commonly used for the inexpensive production of microdevices. One particularly useful approach is described in detail in Dittami et al., Journal of Microelectromechanical Systems, 2008, 17, pp. 850-862. The cytometer can be readily integrated as part of a microchip by fabrication using standard semiconductor manufacturing techniques. Such an approach allows for a number of units and devices to be placed on a common platform for creation of simple off-the-shelf products.

Generally, the cytometer can be formed by patterning electrodes on a substrate through approaches such as shadow-masking and photolithographic patterning. In the former approach, the substrate is covered with a “stencil” pattern exposing areas where electrodes are desired. Using sputter or evaporative deposition approaches, metal is deposited in the exposed areas and blocked from other areas by the stencil. In the second approach, metal is uniformly deposited on the substrate with sputter or evaporative deposition techniques. The metal is subsequently patterned to form electrodes by (i) depositing a photosensitive polymer over the metal, (ii) selectively removing areas of the polymer to expose the underlying metal using light exposure and resist solvents, and (iii) subsequently etching/removing the exposed metal to generate the pattern. Next the fluidic layer can be formed over the electrodes through photolithographic patterning of “permanent” or chemically-resistant photoresists or through thick film technologies such as the bonding of micromolded polydimethylsiloxane to the substrate, adhesive bonding of patterned films or thermal bonding of patterned films. Encapsulation of the fluidic channels is typically accomplished with compression seals of elastomeric materials such as PDMS, adhesive materials such as kapton tape, or thermal bonding approaches such as wafer to wafer bonding or heat-staking of thick films.

### EXAMPLE

A microchip was applied to electrically depolarize rat pheochromocytoma (PC12) cells and to simultaneously detect exocytotic catecholamine release amperometrically. Results demonstrate exocytosis was elicited by flowing cells through an electric field generated by a potentiostat circuit in a microchannel, as well as exocytosis triggered by application of an extracellular voltage pulse across. Electrical finite element model (FEM) analysis illustrated that larger cells experienced greater depolarizing excitation from the extracellular electric fields due to the smaller shunt path and higher resistance to current flow in the channel around the cell. Consistent with these simulations, data recorded from cell clusters and large cells exhibited increased release rates relative to data from the smaller cells. Overall, the system was capable of resolving single vesicle quantal release, in the zeptomole range, as well as the kinetics associated with the vesicle fusion process. Analysis of spike population statistics suggested detection of catecholamines from multiple release sites around the cells.

The PC12 cell line derived from a rat pheochromocytoma, a tumor in the rat adrenal gland was used. These cells release catecholamines, principally dopamine, an electroactive molecule that can be readily detected without electrode functionalization using conventional electrochemical methods. The advantages of PC12 cells for exocytosis studies have been well documented by prior researchers.

Cells were cultured in suspension in 75 mm² tissue culture flasks with RPMI 1640 (Invitrogen, Carlsbad, Calif.) cell culture media supplemented with 15% horse serum (Invitrogen), 2.5% fetal bovine serum (Invitrogen) and 0.02 mg/ml gentamicin (Invitrogen). Flasks were kept in an incubator at 37° C. with 5% CO₂. Media was refreshed every 3-4 days, retaining 10% of the existing media each time. Cells were mechanically agitated to break up cell clusters through repeated washing through a pipettor tip. Additional separation was achieved through weekly, overnight treatment of the cells in 1 mM ethylene glycol tetraacetic acid (EGTA, Sigma-Aldrich, St. Louis, Mo.) with subsequent washing and trituration. Cell media was supplemented with 5 μM dexamethasone (Sigma-Aldrich) to differentiate the cells into more chromaffin-like cells in an effort to increase quantal yield and release efficiency.

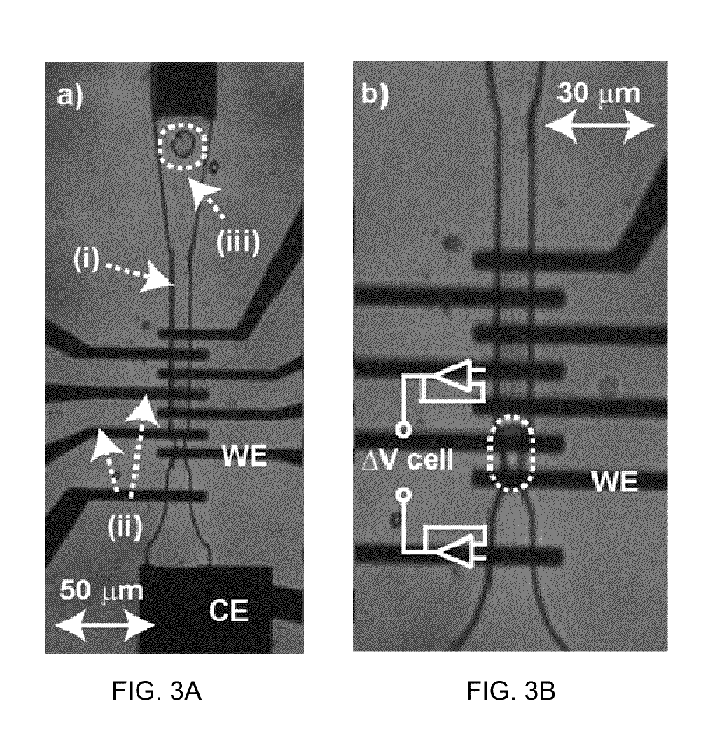

Prior to use, cells were transferred to Locke's buffer consisting of 154 mM NaCl, 5.6 mM KCl, 3.6 mM NaHCO₃, 2.3 mM CaCl₂, 5.6 mM glucose, and 5 mM Hepes (pH 7.4). An aliquot of the solution was immediately transferred to the MEMS device via manual pipette aspiration. Cells were moved back and forth in the channel through application of vacuum or pressure using 2 ml Pipette Pumps (Bel-Art, Pequannock, N.J.) connected through the previously described device interface. Briefly, this interface provided electrical and fluidic interconnects to the device through a polycarbonate headstage providing a “cartridge-based” approach for rapid exchange of individual chips. The fluidic component consisted of ports that mated with the fluidic wells of the microchip on one end and to standard compression fittings on the other end to allow for connection to commercial laboratory tubing and external pumps/vacuums. The interface additionally provided the ability to dynamically view the cells in the chamber with an inverted microscope (IM35, Zeiss, Thornwood, N.Y.). Manual control of 2 ml pipette pumps in conjunction with this direct visual feedback enabled exact positioning of the cell over the desired WE in a static or no-flow condition (as shown in FIG. 3). The pipette pumps were also used to generate the vacuum pressure necessary for channel flow in the flow-through cell experiments.

Three-dimensional (3-D) finite element models (FEM) of PC12 cells in the recording chamber were generated to estimate perturbations in cell membrane potential induced by the inhomogeneous extracellular electric field (Comsol Multiphysics, AC/DC Module, Burlington, Mass.). Simulations used a 10 μm×10 μm cross-sectional area channels to match the microchips. Two cell sizes were simulated, a small 8 μm diameter sphere positioned in the center of the channel, and an 18 μm diameter cell squeezed into the microchannel with a 100 nm saline shunt path between the cell and the channel walls at its tightest point. Dielectric properties were estimated from analogous rat adrenal gland cells. The cell plasma membrane was modelled using a distributed dielectric layer comprised of an area specific conductivity of 2.85 S/m² and a relative permittivity of 26. The cell cytoplasm was modelled with a relative permittivity of 52 and a conductivity of 1.5 S/m. No electrode double-layer effects were modelled as the emphasis was on the voltage differential across the cell in the microchamber. The surrounding cell media was modelled with a relative permittivity of 78 and conductivity of 1.4 S/m (e.g. saline).

Microchips were fabricated at the Utah Nano-Science and Engineering Laboratories at the University of Utah using surface micromachining techniques. Patterned electrodes were formed through photolithographic patterning. In this approach, metal was uniformly deposited on the substrate with sputter deposition. The metal was subsequently patterned to form electrodes by (i) depositing a photosensitive polymer over the metal, (ii) selectively removing areas of the polymer to expose the underlying metal using light exposure and resist solvents, and (iii) subsequently etching/removing the exposed metal to generate the pattern. The fluidic layer was formed over the electrodes through photolithographic patterning of SU-8™ 2010 photoresist (Microchem Corp, Newton, Mass.). A polydimethyl siloxane gasket was used to encapsulate/seal the fluidic channels through compression.

A photomicrograph of the microchip is shown in FIG. 3A. FIG. 3A shows a close-up view of the recording chamber as a PC12 cell (FIG. 3A, (iii)) is pulled into it. The chamber is formed as the 10 μm (depth) fluidic channel (FIG. 3A, (i)) channel narrows to a 10 μm width over an interdigitated electrode array of eight independently addressable gold (Au) electrodes (FIG. 3A, (ii)). The cell is immobilized over the working electrode (FIG. 3A, WE) by a ˜5 μm constriction in the channel as shown in FIG. 3B (dotted ellipse).

Neurotransmitter release from the PC12 cells was measured using a three electrode arrangement with a Au WE (FIGS. 3A and 3B, WE), a Au counter electrode (FIGS. 3A and 3B, CE) and silver/silver chloride (Ag/AgCl) reference electrode (RE) with all controlled by a NPI VA-10 potentiostat (National Precision Instruments (NPI), Tamm, Germany). In a subset of the experiments, individual electrodes on either side of the cell were wired to high-impedance voltage following op-amps (OPA356, Texas Instruments, USA) for passively measuring the voltages in the channel. These outputs were passed to a differential amplifier (DPA-2FX, NPI) to determine the voltage differential across the cell as schematized in FIGS. 3A and 3B. The specific electrode used as the WE was varied between experiments, but the relative positions of the three electrodes were maintained. Prior to each experiment the WE was electrochemically cleaned by cycling the electrodes from −300 mV to +1.5 V (vs Ag/AgCl) in a 0.1 M sulfuric acid (H₂SO₄, Sigma-Aldrich) solution until stable voltammograms were obtained. For chronoamperometry, the WE was held at a potential of +700 mV (vs Ag/AgCl) to ensure oxidation of any released catecholamines. The low-pass filtered (3 kHz, 2-pole Bessel) voltage (current-converted) output of the VA-10 was passed through two additional low-pass (3 kHz) Bessel filters (LPBF-01G and BF-48DGX, NPI) creating an effective 10-pole Bessel filter and scaling amplifier gain of 10 in the third stage. For additional electrical stimulation, or triggering, of neurotransmitter release, two large, axially-positioned electrodes (not shown) were used to apply a mirrored, 5 s voltage pulse across the cell with the electrode apical to the cell set to +800 mV and the electrode basal to the cell set to −800 mV. These relatively large voltages were applied to account for the high-impedance of the electrode double layers. Because of these impedances, fluid voltages in the channel would typically be more than an order of magnitude less than the applied voltage.

Dynamic control of the instrumentation was achieved through GPIB (IEEE-488; National Instruments) connections linked to custom software (IGOR Pro; Wavemetrics, Lake Oswego, Oreg.). All analog signals were sampled with a 16bit A/D converter (ITC-1600, Heka, Bellmore, N.Y.) at 5 kHz.

For off-line data analysis, the WE current was digitally band-pass filtered between 5 Hz and 250 Hz using a sliding Hanning window. Quantal release times were identified by thresholding at a level five times the standard deviation of the baseline noise. Once quantal event times were identified, the release parameters of half width (t_{1/2}), peak height, and quantal molar quantity were calculated directly from the raw unfiltered data. The number of molecules of transmitter released (N, used for determining molar quantity) was calculated by applying Faraday's law, Q=nFN, where n was the number of electrons transferred per molecule (assumed to be 2 for catecholamines), F was Faraday's constant (96,485 C/mol) and Q was the total charge per spike determined by integrating the area under each current spike. Goodness of fit comparisons of single versus double-Gaussian fits of the data were performed by comparing root mean squared deviations (RMSD) values of the fits to the actual data.

Results and Discussion

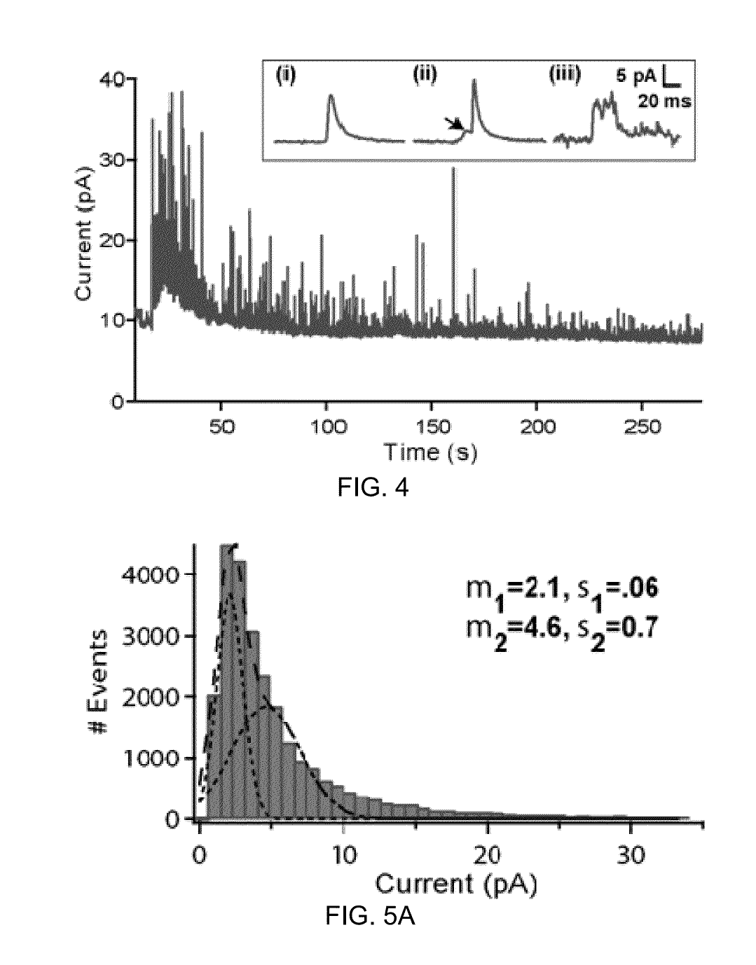

PC12 cells and PC12 cell clusters with dimensions on the order of the channel or larger (i.e. FIGS. 3A and 3B) spontaneously released catecholamines upon positioning over the WE. FIG. 4 shows a typical record for a cluster of six cells in the channel. Individual release events or “quanta” from the cells were clearly resolvable with the microchip. A representative quantal release event is shown in FIG. 4, Inset (i). Detection was sensitive enough to occasionally resolve “foot” events (FIG. 4, Inset (ii)) and “flicker” events (FIG. 4, Inset (iii)) that are presumed to be associated with fusion pore formation and transient release through a fusion pore without full vesicle fusion, respectively. Initial release typically resulted in a large burst of spikes and a concomitant rise in the baseline current level. These data are interpreted as the transient increased vesicle release rate of the readily releasable intracellular vesicle pool and an overall increase in the chamber catecholamine concentration, respectively.

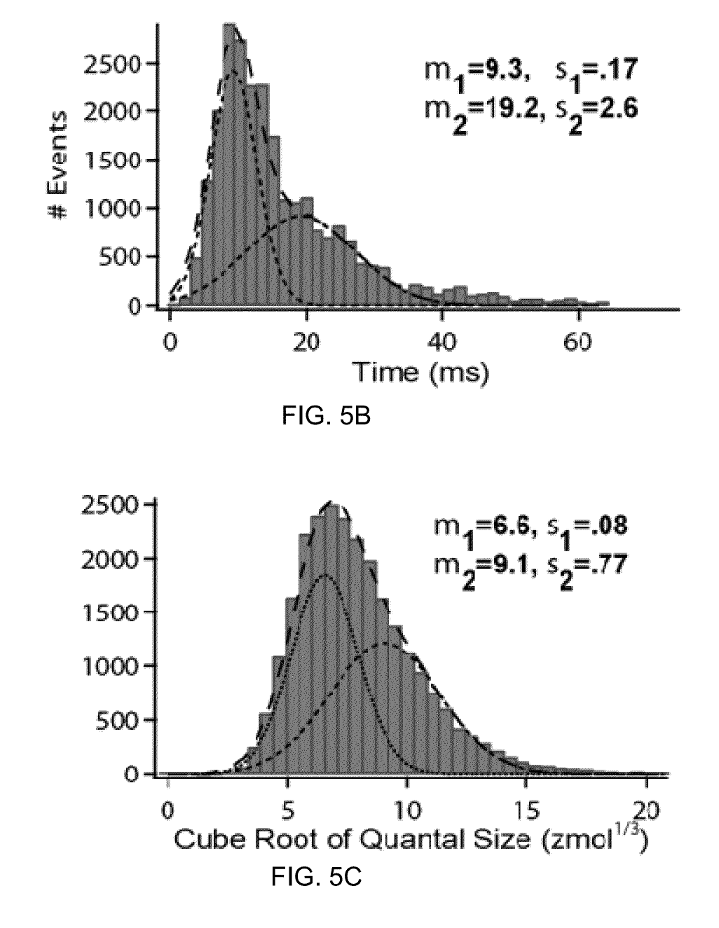

Spike analysis was performed for a population of cells (n=209 cells, k=25,463 spikes). FIG. 5A through 5C show histograms of the key parameters of quantal release including: a) current, b) spike half-width t_{1/2}, and c) cube root of quantal molar quantity. Examination of RMSD values of the fits reveal that all three parameters are significantly better fit with double Gaussian functions than with single Gaussian functions. Unlike their undifferentiated counterparts, dexamethasone-treated PC12 cells are considered to have a homogenous population of vesicle sizes. As a result, the cube root of the quantal molar quantity (which is presumed proportional to vesicle radius cubed) should provide a Gaussian distribution, assuming uniform intra-vesicle biochemical concentration. Consequently, the emergence of two distinct populations of release events in this data indicated the system detected release events originating from multiple sites around the cell. One pool would represent the immediately oxidized catecholamine released at the site in wet contact with the WE and the other pool representing release from more remote regions of the cell. Detection of the latter pool was likely facilitated by the confined geometry of the channel which would serve to sequester the catecholamine release to the WE. This is supported by the larger t_{1/2} and smaller peak amplitudes as diffusional effects would result in a broader, flatter spike as the distance from the release site to the electrode was increased. The cube root of quantal yield of 6.6±0.08 zmol^{1/3} corresponds well with existing data for dexamethasone-treated cells (6.7±1.6 zmol^{1/3}). However, even the smaller of the two half-width means of the Gaussian fits (9.3±0.17 ms) was much larger than that previously reported (2.23±0.24 sec). Rather, the value reported here exactly matches that previously reported for undifferentiated cells. The difference cannot be attributed solely to lack of cell contact with the WE as the longer half-widths were witnessed for very large cells and clusters in which direct contact with the WE was assured. One possibility is that the release sites of the cell were in distinct areas of the membrane from that touching the WE. The significantly lower peak height of both distributions (2.2±0.1 pA and 5.2±1.5 pA) as compared to this prior study (31.4±5.6 pA) supports this hypothesis as well, but it is possible that some other factor may have been at play.

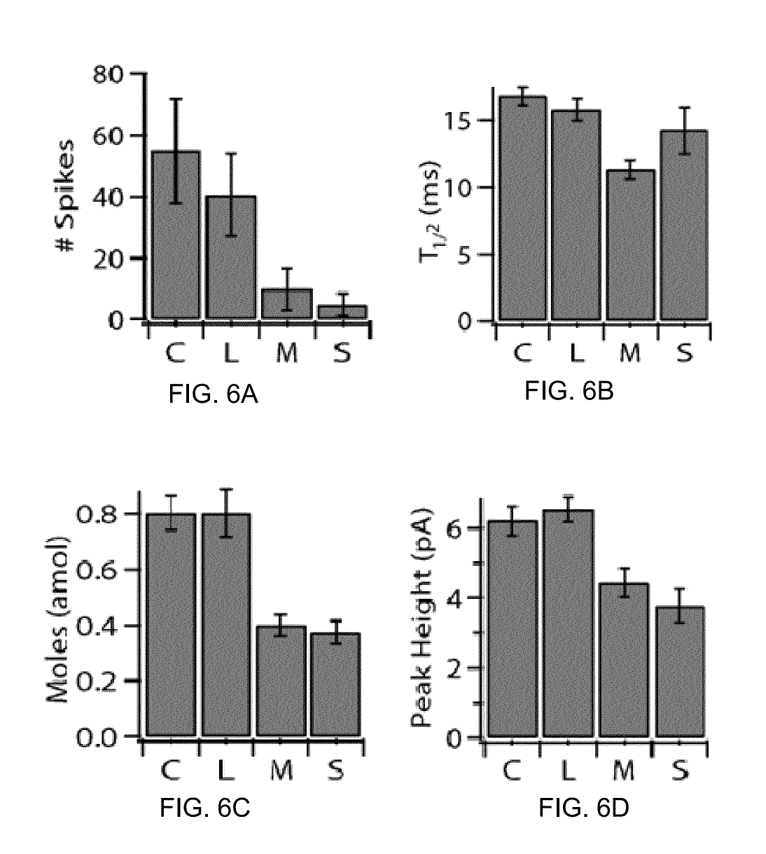

Large cells and clusters of cells generally yielded more quantal recordings. To understand and quantify this, cell size was correlated with: (i) number of spikes released in the first sixty seconds of cell firing, (ii) moles per spike, (iii) t_{1/2} and (iv) peak height. Cells were visually grouped into four size categories: 1) clusters (n=61 samples)—consisting of 2 or more cells in the recording channel. 2) large cells (n=76)—single cells that visually deformed/compressed upon placement over the WE (because they exceeded the channel dimensions). 3) medium cells (n=34)—single cells that were roughly the size of the channel, with no deformation/compression upon entering. 4) small cells (n=10)—single cells with visible gaps between the plasma membrane and the channel wall. As data for unresponsive cells was not incorporated into the analysis, the smaller sample sizes for medium and, particularly, small cells were reflective of the low percentage of these cells having detectable release events. Data is presented in FIG. 6A through 6D in the form of the mean parameter values±one standard error. Statistically significant differences for all parameters were detected between large, medium and small cells (p<0.05, ANOVA). However, for large cells versus cell clusters, the only statistically significant difference was in the number of spikes per cell(s) (p<0.05, T-test). All other parameters showed no statistical differences between the large cells and cell clusters (p<0.05, T-test). As both clusters and large cells should fully occlude the channel, the increase in number of spikes for clusters versus large cells was likely a function of the WE picking up spikes from other cells in the channel. However, the differences in spike numbers among the single cell sizes was most probably a function of the direct correlation between the degree of cell depolarization and the size of the cell, as discussed in detail below. Variation of spike frequency with stimulus strength, as we are reporting here, has also been observed in the exposure of bovine adrenal cells to varying-strength depolarizing solutions. The differences in peak height and t_{1/2} for small and medium cells (p<0.05, T-test) can be explained by diffusional effects. It has been shown that as the WE distance from the cell membrane increases, the spike t_{1/2} values broaden and the peak heights decrease. Given the tightness of fit for larger cells, the seemingly contradictory increase in t_{1/2} for large cells can be explained by the cell deformation in the channel. This deformation results in the cell elongating axially in the channel. As a result, release sites are further separated from the 5 μm electrode and there was an increased diffusion distance for detected spikes from remote regions of the cell. Similarly, for the clusters, we would expect that the contribution of neighbouring cells to result in an increased t_{1/2} and a decreased peak height.

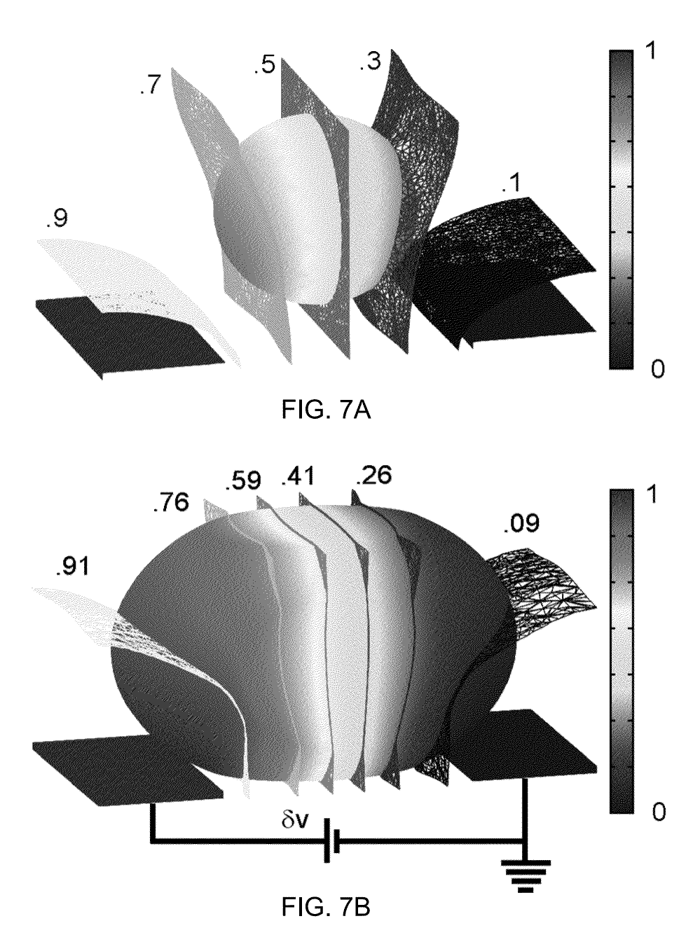

In a previous study it was suggested that mechanical stress can evoke exocytosis from bovine chromaffin cells, which, like PC12 cells, are also derived from the adrenal medula. Although mechanical stimuli may have contributed, it was likely a secondary effect in the present study for two reasons. First, small cells, which experienced no detectable mechanical deformation while in the channel, still exhibited detectable electrically evoked release. Additionally, exposure of a population of cells to cytochalasin-D, a known actin-disrupting agent, did not block the exocytosis activity (data not shown). This result rules out exocytosis evoked by cytoskeletal stretch-activated channels or other mechanical-stress related mechanisms. Instead, release in the present study was primarily evoked by the electric field generated by the potentiostat circuit. FIGS. 7A and 7B show a FEM result illustrating how the extracellular electric field leads to excitation of the cell. Current injected into the channel from the WE flows around the cell to the ground electrode. This causes an extracellular voltage drop across the cell, indicated in the figure using contour surfaces. Since the intracellular voltage is nearly isopotential, perturbation of the extracellular voltage depolarizes the side of the cell near the ground electrode and hyperpolarizes the region near the working electrode. We refer to this technique of using extracellular electrodes to pattern the membrane potential of a cell as micro-domain voltage clamp (μVC). Simulations predicted the voltage drop in the channel across the smaller cell (a) to be ˜0.4 δv versus as compared to ˜0.82 δv for the larger cell (b), where “δv” is the total voltage drop in the fluid media between the two electrodes. Large cells and clusters experience greater depolarizing excitation because of the smaller shunt path and higher resistance to current flow around the cell. Consistent with these simulations, cell clusters and large cells displayed greater release rates than the small cells (FIG. 6A).

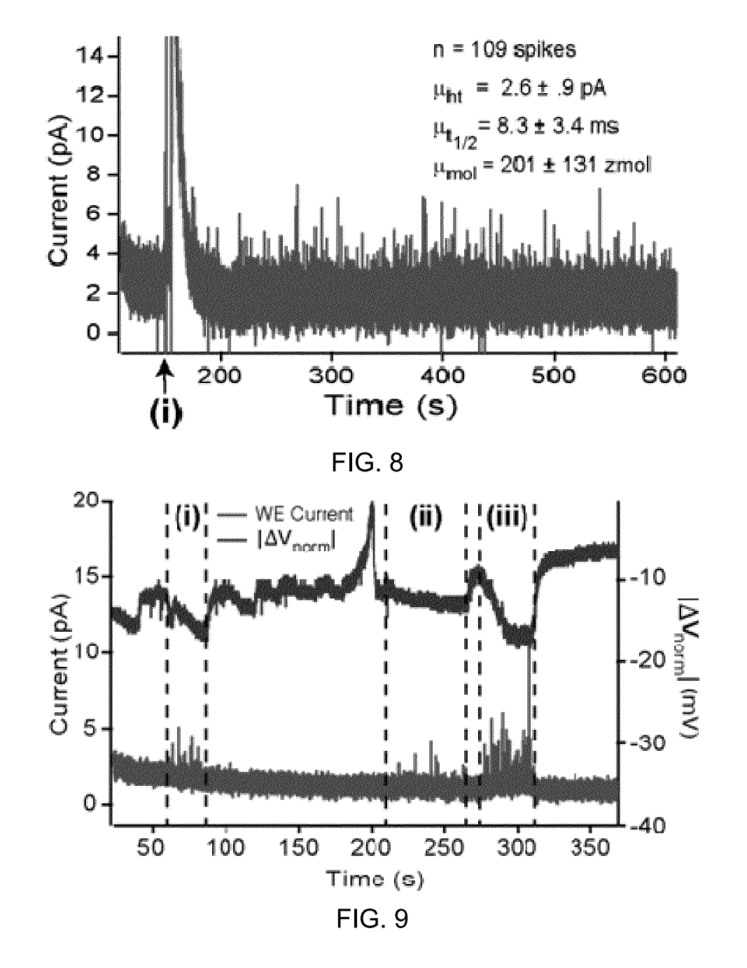

In a subset of experiments we used two additional electrodes placed upstream and downstream of the cell to apply additional excitatory electrical stimuli. One example is shown in FIG. 8. Initially, there was no spontaneous exocytosis from this cell. Application of a ±800 mV, 5 s square pulse on the metal electrodes (at 150 s into the record) axially across the cell evoked a maintained catecholamine release. This triggered release was replicated in 11 cells. However, not all cells showed release even after application of an axial voltage pulse. One likely reason is that smaller cells would require a very large extracellular pulse to realize a sufficiently large depolarizing voltage to evoke release. Because the majority of the voltage would drop across the high-impedance gold electrode double-layer, the magnitude of voltage necessary to depolarize the very loose-fitting cell would exceed the “water window” or voltage at which the decomposition of water occurs. As a result, these voltages, and therefore stimulation, would not be realizable for the smallest cells. Additionally, the current densities required would often result in damage to the microelectrodes. Because the larger cells for the most part would fire “spontaneously” with the potentiostat electric field, the resulting subset of cells that were large enough to be depolarized by the triggered pulse and yet small enough to avoid spontaneous release would be expectedly small.

One potential advantage of the present approach is the possibility for exocytosis flow cytometry and high-throughput. The ability to have the potentiostat in the chronamperometric circuitry serve as both the recording and stimulating mechanism lends itself towards prospective automation. FIG. 9 provides one example of chronoamperometric detection from three different cells as they successively flowed through the recording chamber. The lower trace shows the chronoamperometric current response at the WE and the upper trace shows the recorded channel voltage differential across the cell using the wiring arrangement shown in FIG. 3B. As each cell crossed the WE, quantal release events were observed. The magnitude of the voltage differential and the spike intensity were reflective of the relative cell sizes with the largest cell (iii) producing the greatest response and the smallest cell (ii) the least response.

Improved selectivity for dopamine over the most physiologically-relevant interferent, ascorbic acid, could be achieved by coating the WE with a cation-selective membrane such as Nafion. Alternatively, more descriptive chemical identification of vesicle contents can be achieved with cyclic voltammetry. Additionally, it was observed that the mean spike amplitude for a particular WE on a given microchip would decrease with time (data not shown). This was most likely due to the reduction in electron transfer efficiency due to the build-up of poly-dopamine on the WE but could also have been due to non-specific adhesion of cellular constituents and extracellular proteins. For large populations of cells, a mechanism for periodic cleaning of the electrodes, for example with cycling in sulfuric acid can be used.

We have presented a microchip capable of electrically evoking and electrochemically resolving quantal release events from single cells and cell clusters. Overall detection of catecholamine release from PC12 cells was comparable to the conventional CFE electrochemical techniques of release detection with our ability to resolve quantal release events and even subcomponents of vesicle fusion in the exocytosis process. Advantages of the present approach are the simplicity in which cells are loaded using device microfluidics, automated positioning of the electrodes through photolithographic patterning, and exocytosis flow cytometry. Additionally, more rapid studies of populations of cells have the potential to facilitate discovery through statistical observations of rare events such as the formation of the fusion pore and/or flicker events.

The foregoing detailed description describes the invention with reference to specific exemplary embodiments. However, it will be appreciated that various modifications and changes can be made without departing from the scope of the present invention as set forth in the appended claims. The detailed description and accompanying drawings are to be regarded as merely illustrative, rather than as restrictive, and all such modifications or changes, if any, are intended to fall within the scope of the present invention as described and set forth herein.

What is claimed is:

1. A method of triggering cellular exocytosis events and measuring quantal release of electroactive chemicals, comprising:
a) flowing a cell through a single cell channel having a pair of electrodes oriented to direct current through the cell at a stimulation location along the single cell channel, wherein the pair of electrodes includes a working electrode and a counter electrode, and the single cell channel having dimensions which provide direct contact of the cell with walls of the single cell channel sufficient to substantially reduce or eliminate shunt paths around the cell at the stimulation location;
b) exposing the cell to an electric field at the stimulation location using the pair of electrodes sufficient to trigger exocytosis;
c) measuring current entering the working electrode; and
d) correlating the changes in the measured current with a quanta of the electroactive chemicals.

2. The method of claim 1, wherein the flowing allows for a temporary pause of the cell at the stimulation location during the exposing.

3. The method of claim 1, wherein the flowing is continuous.

4. The method of claim 1, wherein the dimensions have a cross-sectional diameter equal to or less than a free diameter of the cell.

5. The method of claim 4, wherein the cross-sectional diameter is from about 60% to about 90% of the free diameter.

6. The method of claim 1, wherein the dimensions provide substantially complete fluidic isolation of the working electrode from the counter electrode when the cell is at the stimulation location.

7. The method of claim 1, wherein the counter electrode is oriented downstream from the working electrode.

8. The method of claim 1, wherein the counter electrode and the working electrode are electrically connected to a potentiostat circuit and further comprising a reference electrode oriented in the single cell channel.

9. The method of claim 1, further comprising at least one supplementary working electrode oriented upstream of the working electrode.

10. The method of claim 1, wherein the electric field is a pulsed electric field.

11. The method of claim 1, wherein the electric field has an applied potential sufficient to depolarize the cell but below a water window of the cell.

12. The method of claim 1, wherein the electroactive chemicals are neurotransmitters.

13. The method of claim 12, wherein the neurotransmitters are selected from the group consisting of dopamine, serotonin, epinephrine, norepinephrine, histamine, DOPAC, HVA, nitric oxide, ascorbic acid, 5-HIAA, and combinations thereof.

14. The method of claim 1, further comprising cleaning the working electrode through potential cycling of the electrode or exposure to a mild etch solution.

15. The method of claim 1, wherein the pair of electrodes are configured for both electrochemical stimulation and measurement of exocytosis events.

16. The method of claim 1, wherein a free diameter of the cell is from about 5 μm to about 30 μm.
